(19) United States
(12) Patent Application Publication
(10) Pub. No.: US 2005/0281850 A1
(43) Pub. Date: Dec. 22, 2005

(54) Cosmetic/dermatological inverse emulsions containing calcitriol and clobetasol 17-propionate

(75) Inventors: Leslie Zanutto, Paulhaguet (FR); Sandrine Orsoni, Mandelieu (FR); Laurent Fredon, Roquefort Les Pins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A.

(21) Appl. No.: 10/944,887

(22) Filed: Sep. 21, 2004

(30) Foreign Application Priority Data
Jun. 17, 2004 (FR) 04/06612

(51) Int. Cl. A61K 31/59; A61K 7/00

(52) U.S. Cl. 424/401; 514/167; 514/171

(57) ABSTRACT

Topically applicable cosmetic/dermatological compositions useful for the treatment of disorders of the skin, notably psoriasis, comprise inverse emulsions containing a glycol or water-glycol dispersed hydrophilic phase, a lipophilic continuous phase, an emulsifier having an HLB of between 2 and 7 and also having calcitriol and clobetasol 17-propionate dissolved therein.

### CROSS-REFERENCE TO PRIORITY APPLICATION

This application claims priority under 35 U.S.C. § 119 of FR-04/06612, filed Jun. 17, 2004, hereby expressly incorporated by reference and assigned to the assignee hereof.

### BACKGROUND OF THE INVENTION

1. Technical Field of the Invention

The present invention relates to novel compositions comprising inverse emulsions containing two dissolved active agents, calcitriol and clobetasol 17-propionate, to a variety of cosmetic and dermatological applications thereof and to a process for the preparation of same.

2. Description of Background and/or Related and/or Prior Art

Human skin consists of two compartments, namely a surface compartment, the epidermis, and a deep compartment, the dermis.

The dermis gives the epidermis a solid support. It is also the epidermis' nourishing factor. It consists mainly of fibroblasts and of an extracellular matrix which is itself composed mainly of collagen, elastin and a substance known as ground substance. Leukocytes, mastocytes and tissue macrophages are also found therein. It also contains blood vessels and nerve fibers.

The epidermis is in contact with the external environment. Its role is to protect the body against dehydration and external attack, whether chemical, mechanical, physical or infectious.

Natural human epidermis is composed mainly of three types of cells: keratinocytes, which form the vast majority, melanocytes and Langerhans cells. Each of these cell types contributes, by virtue of its intrinsic functions, towards the essential role played in the body by the skin.

The cells constituting the epidermis are delimited by a lipid domain. The epidermal lipids are synthesized mainly in the live epidermis. They consist essentially of phospholipids, sphingolipids, cholesterol, free fatty acids, triglycerides, cholesterol esters and alkanes. During cell differentiation, the phospholipids, whose role consists in developing the fluid structure of the cell membranes of the live layers of the epidermis, are gradually replaced by a mixture predominantly composed of fatty acids, cholesterol and sphingolipids, which are essential constituents of the corny layer of the epidermis (stratum corneum).

The lipids of the intercorneocytic cement of the skin, and especially the ceramides, are organized into lamellar bilayers or leaflets and contribute towards the cohesion of the stratum corneum so as to maintain the integrity of the barrier and its protective, anti-penetration and anti-irritation role especially.

Many active agents present the difficulty of being very sparingly soluble in the cosmetic or pharmaceutical solvents commonly used, especially water, and sensitive to an aqueous environment. This water sensitivity may lead to chemical instability of the active agent and/or to crystallization of the active agent initially dissolved. This water sensitivity thus limits their formulation in cosmetic or dermatological compositions applied topically or orally.

The phenomena of chemical degradation and/or crystallization of the active agent in the presence of water have the consequence of reducing the efficacy and creating uncertainty as regards the dose of active agent engaged during its use, which runs counter to the desired objective. In addition, this degradation and/or crystallization of the active agent can modify the overall stability of the compositions, and also their appearance.

The galenical form most commonly used at the present time in dermatology is an oil-in-water emulsion in which the active agent is preferably dissolved in the lipophilic phase. However, this solution remains relatively unsatisfactory, since it can lead to products that are not particularly pleasant to use, due to their physically unstable, greasy, tacky feel.

Another possibility is to dissolve the active agent in the outer hydrophilic phase of the emulsion, in the limit of its solubility in aqueous or water-glycol media. However, this solution does not make it possible to solve the chemical stability problems encountered, since the water activity of the emulsion remains very high.

The replacement of all or some of the aqueous phase with one or more glycols would lead to cosmetically unacceptable formulations. The reason for this is that it is known to those skilled in the art that beyond 20% glycol in the outer phase, the formulation is cosmetically unacceptable because of its tacky feel, and its physical stability would not be ensured.

The development of an inverse emulsion (the term “inverse emulsion” means an emulsion of the type: hydrophilic phase dispersed in lipophilic phase) as an alternative was not obvious to those skilled in the art given the known difficulties of formulating active agents that show chemical instability and/or crystallization problems in water. Furthermore, in WO 03/011243 and FR-2,850,575, assigned to the assignee hereof, glycol-in-silicone formulations are described whose viscosity is occasionally found to be relatively unstable. Specifically, the flow threshold of the formulation can decrease as a function of time and temperature, like the majority of emulsions with an oily continuous phase. One skilled in the art, therefore, would not seek this type of composition to solve the present problem of formulation of the two active agents.

The use of hydrophilic solubilizers, for instance propylene glycol, was also not apparent to those skilled in the art, given that the high concentrations required were not favorable towards good physical stability of the formulation and towards an acceptable cosmetic feel.

Obtaining good tolerance with solubilizers such as propylene glycol was also not obvious since skin intolerance phenomena has been shown in humans, for example in healthy humans (Motoyoshi et al., Cosmet and Toiletries, 99, 83-89,1984).

However, it is important for the compositions according to the present invention, hereinafter more fully described, to incorporate a large proportion of glycol in order to promote the action of the corticoid and thus lead to good vaso-constriction.

Moreover, the combination of active principles is not administered in a conventional manner in the treatment of dermatological complaints, conditions or afflictions. The difficulties mainly encountered by those skilled in the art during the combination of two active principles are the problems of chemical instability and the interactions that the active principles might show when they are present within the same formulation.

Very few treatments thus exist combining calcitriol and a corticoid. Specifically, vitamin D and its derivatives are unstable in aqueous media, and sensitive to acidic pHs, while corticoids and more particularly clobetasol propionate are, themselves, sensitive to basic media. It was therefore not obvious to one skilled in the art to combine and stabilize within the same composition an active agent of vitamin D type and a corticosteroid.

Calcitriol is a vitamin D analogue used to regulate the level of calcium in the body. Its use in the treatment of dermatological diseases has been described especially in U.S. Pat. No. 4,610,978 for the treatment of psoriasis. This '978 patent suggests compositions comprising calcitriol, which may also contain an amount of an anti-inflammatory agent such as a corticosteroid; however, no concrete embodiment of a combination of calcitriol and of corticosteroid is described or tested in terms of efficacy.

FR-2,848,454, assigned to the assignee hereof, describes that the combination of calcitriol with a corticosteroid makes it possible to obtain a synergistic effect in the treatment of certain dermatological complaints such as psoriasis, atopic dermatitis, contact dermatitis and seborrhoeic dermatitis, without, however, proposing stable pharmaceutical compositions combining the two active agents.

Moreover, in the field of dermatology and of formulation of pharmaceutical compositions, one skilled in the art is led to seek compositions which, not only must be physically and chemically stable, but also must make it possible to release the active agent and to promote its penetration through the layers of the skin in order to improve its efficacy.

Thus, serious need continues to exist for compositions that can satisfy one or more of the following aspects: formulate two active agents within the same composition, have good stability of the formulation to cold and to heat, in particular as regards the maintenance of the size of the globules and the absence of phase separation and especially good stability of the viscosity as a function of time, have good resistance of the active agents with respect to oxidation phenomena, allow good chemical stability of the active agents and good availability of the said agents to the skin, and show good skin tolerance. It is moreover useful for the preparation of such compositions to benefit from an advantageous preparation method.

### SUMMARY OF THE INVENTION

Surprisingly, formulations of glycol-in-oil type have now been developed that make it possible to overcome the various problems associated with the aspects mentioned above, by especially providing good physical stability of the composition per se, but also to allow good chemical stability and availability of the active agents it contains. The composition according to the invention also have the advantage of having good skin tolerance, of allowing a high dispersed fraction by volume, and of having a high content of glycol, providing good vasoconstriction.

The present invention thus features compositions containing two dissolved active agents, a vitamin D derivative, namely calcitriol, and a corticoid, namely clobetasol 17-propionate (also referred to hereinbelow as clobetasol propionate). The compositions according to the invention are characterized in that they are inverse emulsions containing a glycol or water-glycol dispersed hydrophilic phase, a lipophilic continuous phase and an emulsifier having an HLB of between 2 and 7.

The term “HLB” means the Hydrophilic/Lipophilic Balance, which corresponds to the equilibrium between the size and strength of the hydrophilic group and the size and strength of the lipophilic group of the emulsifier.

This invention also permits good release/penetration of the active agent into the various layers of the skin, providing good availability of the active agent in the skin, said active agent being employed in dissolved form.

The term “dissolved form” means a dispersion in molecular form in a liquid, no crystallization of the active agent being visible to the naked eye or even an optical microscope in cross polarization.

The present invention thus also features preparing inverse emulsions, containing a glycol or water-glycol hydrophilic phase, which are entirely stable (globule size and viscosity), even at a high dispersed fraction by volume, showing no chemical degradation and/or crystallization of the active agents.

The present invention also features the preparation of inverse emulsions containing an active agent dissolved in the lipophilic phase of the emulsion, and showing good physicochemical stability, and no crystallization of the active agent.

The present invention thus features compositions comprising, in a physiologically acceptable medium:
(a) calcitriol in dissolved form;
(b) clobetasol 17-propionate in dissolved form;
(c) at least one glycol;
(d) an emulsifier having an HLB of between 2 and 7;
(e) a small or minor amount of water;
(f) a lipophilic phase.

The compositions according to the invention are preferably suitable for topical application onto the skin, the integuments and/or mucous membranes. They generally contain a physiologically acceptable medium and an amount of active compound that is sufficient to elicit the desired effect.

### DETAILED DESCRIPTION OF BEST MODE AND SPECIFIC/PREFERRED EMBODIMENTS OF THE INVENTION

Advantageously, the compositions according to the invention comprise from 0.0001% to 0.1% by weight and preferably from 0.001% to 0.05% by weight of clobetasol 17-propionate, relative to the total weight of the composition. The compositions that are preferred according to the invention more particularly comprise from 0.025% to 0.05% by weight of clobetasol propionate relative to the total weight of the composition.

In one preferred embodiment according to the invention, the clobetasol 17-propionate is dissolved in an inner glycol or water-glycol phase.

Advantageously, the compositions according to the invention comprise from 0.00001% to 0.1% by weight, preferably from 0.0001% to 0.001% by weight and more preferably from 0.0002% to 0.0005% by weight of calcitriol relative to the total weight of the composition. The compositions according to the invention more particularly comprise 0.0003% by weight of calcitriol relative to the total weight of the composition.

It is advantageously possible to add it to the dispersed phase.

In one preferred embodiment according to the invention, the calcitriol is dissolved in a solvent such as an alcohol or an oil, present in concentrations of from 0 to 10% by weight relative to the total weight of the formulation.

As alcohol that may be used as solvent for the calcitriol according to the invention, a linear or branched C₁ to C₄ alcohol, of ethanol or isopropanol type, is intended. The alcohol that is preferred according to the present invention is ethanol.

As oil that may be used as solvent for the calcitriol according to the invention, an oil of the caprylic/capric triglyceride type (Miglyol 812) is preferably intended.

The glycols according to the present invention may be defined as alkylene glycols or polyalkylene glycols. Non-limiting examples that may be mentioned include alkylene glycols and polyalkylene glycols (C1 to C6) such as ethylene glycol, polyethylene glycol (2 to 20 monomers), propylene glycol, dipropylene glycol, butylene glycol, pentylene glycol and hexylene glycol. They may or may not be oxyethenylated (2 to 50 EO). The ones that are preferred according to the invention are hexylene glycol, propylene glycol, dipropylene glycol and polyethylene glycol 400 (PEG 400).

The glycols that may be used according to the invention will advantageously have as solubility parameter a δp of less than 10, it being understood that the three Hansen solubility parameters: δd, δp and δh characterize, for a given constituent, the energies corresponding, respectively, to the dispersive inter-actions, polar interactions and interactions of hydrogen bonding type existing between the molecules of this constituent, δp more particularly characterizing the Debye interaction forces between dipoles and being a function of the number of oxygen atoms in the formula of the given constituent (S. Paint Technology, 30,195,1967, “The three dimensional solubility parameter—Key to paint component affinities”).

The volume fraction of the hydrophilic phase dispersed in the emulsion according to the invention ranges from 10% to 90% relative to the total volume of the emulsion. It may be exclusively glycol-based or water-glycol-based. The proportion of glycols by volume (relative to the total volume of the dispersed phase) is from 10% to 100%, for example from 30% to 100% and in particular from 60% to 100%.

From 30% to 50% of glycols (proportion relative to the total volume of the dispersed phase) will preferably be used for cosmetic applications.

According to the invention, the weight proportion of glycols relative to the total weight of the composition is from 20% to 90% and preferably from 30% to 60%.

As lipophilic compounds that may be used to constitute the continuous lipophilic (or fatty) phase of the emulsions according to the invention, mention may be made of mineral oils (liquid paraffin), oils of plant origin (avocado oil, soybean oil), oils of animal origin (lanolin), synthetic oils (perhydrosqualene), silicone oils (cyclomethicone, dimethicone) and fluoro oils (perfluoropolyethers). Fatty alcohols such as cetyl alcohol, Guerbet alcohols, in particular octyl-dodecanol, which is known under the name Eutanol G, fatty acids, waxes and gums, and in particular silicone gums, may also be used.

The fatty phase may also be linear or branched monoesters, diesters or triesters of synthetic origin, in particular isopropyl myristate or palmitate, or caprylic/capric triglyceride (Miglyol 812).

Preferably, non-oxidizable compounds are used to make up the oils of the continuous lipophilic phase, which will preferably be chosen from those of silicone type, those of ester type and those of mineral type.

Preferentially, the compounds constituting the lipophilic phase of the composition according to the invention are mineral oils and especially liquid paraffin, triglycerides of the caprylic/capric triglyceride type sold under the name Miglyol 812, esters and preferentially cetearyl isononanoate sold under the name Cetiol SN, and silicone oils, more particularly cyclomethicones, used alone or as a mixture.

Advantageously, the proportion of the lipophilic phase, by weight relative to the total weight of the composition, is from 5% to 60% and more particularly from 10% to 25%.

It is also possible to characterize a preferred embodiment of the invention by referring to the water activity (aw) of the hydrophilic phase in the compositions according to the invention.

The invention also relates particularly to a composition as defined above, characterized in that the water activity aw of the hydrophilic phase is less than 0.85.

The water activity aw of a medium containing water is the ratio of the vapor pressure of water of the product “PH₂O product” and of the vapor pressure of pure water “P_{H2O}pure” at the same temperature. It may also be expressed as the ratio of the number of water molecules “N_{H2O}” to the total number of molecules “N_{H2O}+N_{dissolved substances}”, which takes into account that of the dissolved substances “N_{dissolved substances}”.

It is given by the following formulae:

$${a}_{w} = \frac{{P}_{H20} product}{{P}_{H20} pure} = \frac{{N}_{H20}}{{N}_{H20} + {N}_{dissolved substances}}$$

Various methods may be used to measure the water activity a_{w}. The most common is the manometric method, via which the vapor pressure is measured directly.

A cosmetic or dermatological composition conventionally has a water activity of about from 0.95 to 0.99. A water activity of less than 0.85 represents an appreciable decrease.

Advantageously, the compositions according to the invention comprise a small proportion of water, i.e., from 1% to 30% and preferably from 10% to 20% water relative to the total weight of the composition.

For the preparation of the inverse emulsions according to the invention, the presence of emulsifiers is obligatory. The emulsifiers (or surfactants) are natural or synthetic substances formed from a hydrophilic or polar portion and a lipophilic or polar portion. They are amphiphilic molecules, since they have a double polarity. Emulsifiers are characterized by their HLB: if the HLB is high, the hydrophilic portion is predominant, if the HLB is low, the lipophilic portion predominates.

Among these emulsifiers there are preferably included polymeric emulsifiers, which are characterized by a high molecular mass and a non-linear structure, which allows greater anchoring at the water/oil interface than that obtained with emulsifiers of monomer type.

The emulsifiers that may be used according to the invention, alone or as a mixture, are those that make it possible to prepare inverse emulsions with an HLB of less than 7 and preferably between 2 and 7.

In general, the preferred emulsifiers are silicone emulsifiers, of organopolysiloxane type, such as:
E1) polyalkylmethicone copolyols (optionally crosslinked oxyalkylenated polyalkylmethylsiloxane) containing:
saturated or unsaturated, linear or branched C6 to C20 alkyl chains
a polyoxyethylene unit of 1 to 50 EO (ethylene oxide) and/or
a polyoxypropylene unit of 1 to 50 PO (propylene oxide)
E2) oxyalkylenated polyalkyldimethylmethylsiloxane containing:
saturated or unsaturated, linear or branched C6 to C20 alkyl chains
a polyoxyethylene unit of 1 to 50 EO and/or
a polyoxypropylene unit of 1 to 50 PO.

The organopolysiloxanes of the compositions of the invention especially contain one or more oxyalkylene and in particular oxyethylene (EO) groups, for example from 1 to 40 oxyalkylene units, preferably from 1 to 20, better still from 10 to 20, more preferably from 12 to 20 and even better still from 12 to 18 oxyalkylene units, which can form polyoxyalkylene and especially polyoxyethylene chains. These groups may be pendent or at the end of a chain. The silicon atoms bearing these groups are advantageously from about 1 to 10 and better still from 1 to 6 in number. The silicone structure forming the polymer skeleton of the organopolysiloxane containing oxyalkylene group(s) is advantageously a polydimethylsiloxane (PDMS) structure in which, optionally, some of the methyl groups are substituted with C2 to C30, preferably C8 to C24 and better still C10 to C20 alkyl groups or phenyl groups, which are either at the end of a chain or pendent.

Advantageously, the emulsifiers that will thus be used as emulsifiers of type E1 or E2 are silicone emulsifiers, for instance alkyldimethicone copolyols such as Abil EM-90, or the mixture of dimethicone copolyol and cyclomethicone, sold by Dow Corning under the name 3225C Formulation Aid, the lauryl methicone copolyol sold under the name Emulsifier 10 by Dow Corning, or mixtures based on a silicone polymer, such as the cyclopentasiloxane & PEG/PPG—19/19 dimethicone sold under the name DC BY11-030 by Dow Corning, the cetyl dimethicone copolyol with polyglyceryl-4 isostearate and hexyl laurate, sold under the name Abil WE09 by Goldschmidt, Abil EM 97 from Goldschmidt (dimethicone copolyol & cyclomethicone), Wacker SPG 128 VP from Wacker (cyclomethicone and octyldimethicone methoxy glycosyl), or Silwax WD-IS (dimethicone copolyol isostearate).
E3) Siloxane monoalkyl or polyalkyl esters, for example Silwax S from Lambent (dimethiconol stearate),
E4) alkoxylated carboxylic acid esters, for instance polyhydroxylated PEG alkyl esters, for example Arlacel P 135 from Uniqema (PEG-30 dipolyhydroxy-stearate).

Emulsifiers with an HLB of between 2 and 7 will preferably be used, preferentially a silicone W/O emulsifier with an HLB of between 2 and 7 and preferentially a polymeric silicone W/O emulsifier with an HLB of between 2 and 7.

As a variant, the inverse emulsions of the invention may be advantageously made and stabilized with emulsifiers or combinations of emulsifying nature, such as the combination of an oxyalkylenated crosslinked organopolysiloxane elastomer and a crosslinked and at least partially neutralized poly-(2-acrylamido-2-methylpropanesulfonic acid) polymer.

The preferred emulsifiers according to the invention are cyclopentasiloxane & PEG/PPG-19/19 dimethicone sold under the name DC BY11-030 by Dow Corning, and lauryl methicone copolyol sold under the name Emulsifier 10 by Dow Corning.

The composition according to the invention will especially contain, expressed as weight percentages, from 0.5% to 15% and preferably from 1% to 8% by weight of emulsifier relative to the total weight of the composition.

In one preferred embodiment according to the invention, the composition also comprises “Theological” additives. Surprisingly, these additives, used according to the present invention, make it possible to obtain the desired stability and especially the stability of the viscosity over time and at various temperatures.

Thus, one skilled in this art may add any known rheological additive. However, the rheological additives that are particularly suitable for the desired effect, which are referred to hereinbelow as “viscosity stabilizers” according to the present invention, are especially:
PEG-150 decyl alcohol/SMDI copolymer sold under the trademark Aculyn 44 by Rohm & Haas;
polyglyceryl methacrylate & propylene glycol sold under the trademark Lubrajel CG by Sederma;
the combination of ethylene/propylene/butylene/-styrene copolymer in isododecane, sold under the trademark Versagel MD 1600 by Penreco;
the combination of ethylene/propylene/butylene/-styrene copolymer in isohexadecane, sold under the trademark Versagel MC 1600 by Penreco;
dimethiconol behenate sold under the trademark Mirasil Wax B by SACI;
silicone elastomer and decamethylcyclopentasiloxane sold under the trademark elastomer ST 10 by Dow Corning.

The compositions according to the present invention also preferably comprise at least one electrolyte. As an example of an electrolyte that may be used according to the invention, mention may be made of magnesium sulfate (MgSO₄).

According to one preferred embodiment, the compositions according to the invention also contain antioxidant compounds such as DL-α-tocopherol, butyl hydroxyanisole or butylhydroxytoluene, propyl gallate, superoxide dismutase, ubiquinol or certain metal-chelating agents. The antioxidants preferably used in the compositions according to the invention are DL-α-tocopherol, butylhydroxyanisole and butylhydroxy-toluene.

Moreover, it is possible to combine the main emulsifiers described above with one or more co-emulsifiers having an HLB of greater than 6. The (co-emulsifier/emulsifier) ratio will advantageously be less than 1.5 and preferably less than 0.75. Examples that may be mentioned include: polyoxyethylenated or non-polyoxyethylenated alkyl or polyalkyl esters of sorbitan (for example: sorbitan monolaurate 20 EO or sorbitan monooleate 20 EO (Tween 80 from Uniqema)); polyoxyethylenated alkyl or polyalkyl ethers or esters (ceteareth-20 (Eumulgin B2 from Cognis) or steareth (Brij 78) 20 EO); ethoxylated and esterified alkyl or polyalkyl mono- or polyglucosides (for example, PEG-20 methylglucose sesquistearate (Glucamate SSE-20 from Amerchol)); alkyl or polyalkyl esters or ethers of polyglycerol (for example polyglyceryl-4 isostearate or PEG-8 stearate (Myrj 45)).

In a known manner, the compositions of the invention may also contain adjuvants that are common in cosmetics and dermatology, such as hydrophilic or lipophilic gelling agents, humectants, for instance glycerol and sorbitol, fatty-phase thickeners, preservatives, solvents, fragrances, fillers, screening agents, pigments, odor absorbers, dyestuffs and metal-chelating agents. The amounts of these various adjuvants are those conventionally used in the fields under consideration, for example from 0.01% to 20% of the total weight of the composition. Depending on their nature, these adjuvants may be introduced into the lipophilic phase or into the hydrophilic phase. These adjuvants, and the concentrations thereof, should be such that they do not harm the cosmetic and/or dermatological properties of the compositions according to the invention.

Hydrophilic gelling agents that may be mentioned, in particular, include carboxyvinyl polymers (carbomer), acrylic copolymers such as acrylate/alkyl-acrylate copolymers, polyacrylamides, polysaccharides, natural gums and clays, and lipophilic gelling agents that may be mentioned include modified clays, for instance bentones, metal salts of fatty acids and hydrophobic silica.

The compositions according to the invention have a cosmetically acceptable feel, good skin tolerance, good physical stability, i.e., absence of phase separation and maintenance of the globule size under cold conditions (4° C.) and under warm conditions (45° C.) over a long period, for example over 2 months, especially with a stable viscosity over this period. The composition according to the invention also makes it possible to give the active agents good chemical stability and to avoid their crystallization over time.

In particular, the invention relates to cosmetic or dermatological compositions for topical application onto the skin, the integuments and/or mucous membranes, in the form of an inverse emulsion containing a dispersed glycol or water-glycol hydrophilic phase and a lipophilic continuous phase, comprising, formulated into a physiologically acceptable medium (i.e., a medium that is compatible with topical application to the skin, the integuments and/or mucous membranes), expressed as weight percentages:
(a) from 0.0001% to 1% by weight of calcitriol in dissolved form;
(b) from 0.001% to 0.1% by weight of clobetasol 17-propionate in dissolved form;
(c) from 30% to 100% of glycols;
(d) from 0.5% to 8% of emulsifier with an HLB of between 2 and 7;
(e) from 1% to 30% water;
(f) from 5% to 60% of lipophilic phase.

In one particular embodiment of the invention, the dispersed hydrophilic phase has a water activity of less than 0.85.

The present invention also features compositions that are triple emulsions of hydrophilic phase/lipophilic phase/hydrophilic phase type comprising an outer hydrophilic phase and a lipophilic phase constituting, with an inner hydrophilic phase, an inverse emulsion (“primary inverse emulsion” in the context of this triple emulsion) according to the invention.

Advantageously, the present invention relates to a triple emulsion of hydrophilic phase/lipophilic phase/hydrophilic phase type in which the inner hydrophilic phase of the triple emulsion has a water activity value of less than or equal to 0.85, especially so as to improve the stability of the active agent present in the inner hydrophilic phase.

According to one particular embodiment of the invention, the water activity value of less than or equal to 0.85 is obtained by incorporating an effective amount of glycol. The term “effective amount” means an amount of polyol that is sufficient to obtain a low water activity value, i.e., a water activity value of less than or equal to 0.85.

According to one particular embodiment of the invention, the primary inverse emulsion constitutes from 20% to 35% and more particularly about 25% by weight of the triple emulsion.

The triple emulsion is conventionally prepared by preparing the primary emulsion and incorporating a given amount of the primary emulsion into the outer hydrophilic phase.

The invention also relates to a triple emulsion of hydrophilic phase/lipophilic phase/hydrophilic phase type, comprising an outer hydrophilic phase, a lipophilic phase constituting, with an inner hydrophilic phase, an inverse emulsion (“primary inverse emulsion” in the context of this triple emulsion) according to the invention, which comprises a gelled outer hydrophilic phase containing:
1) at least one emulsifying copolymer comprising a majority fraction of a C₃-C₆ monoolefinically unsaturated carboxylic acid monomer or the anhydride thereof and a minority fraction of acrylic acid fatty ester monomer, and
2) at least one crosslinked poly(acrylamidomethyl-propanesulfonic acid).

Moreover, according to one preferred embodiment of the invention the lipophilic phase of the triple emulsion contains at least one silicone oil and/or a silicone emulsifier.

The emulsifying copolymers that may be used in the triple emulsions according to the present invention are prepared by polymerizing a predominant amount of a monoolefinically unsaturated carboxylic monomer or the anhydride thereof, with a smaller amount of fatty-chain acrylic ester monomer. The term “fatty chain” means a linear or branched alkyl radical containing from 8 to 30 carbon atoms.

The amount of carboxylic monomer or anhydride thereof preferably ranges from 80% to 98% by weight and more particularly from 90% to 98% by weight, while the acrylic ester monomer is present in amounts ranging from 2% to 20% by weight and more particularly from 1% to 10% by weight, the percentages being calculated relative to the weight of the two monomers.

The emulsifying copolymers of the invention are described in EP-A-0,268,164 and are obtained according to the preparation methods described therein.

The emulsifying copolymers that are particularly preferred are those with a viscosity, measured using a Brookfield viscometer in a water solution at 2% and at 25° C., of less than or equal to 5,000 cps (5 Pa.s) and more preferably of about 3,000 cps (3 Pa.s).

An acrylate/C₁₀-C₃₋₀-alkylacrylate copolymer and especially the product sold under the name Pemulen TR 1 by Goodrich is more particularly used.

The emulsifying copolymer is used in the triple emulsion according to the invention in a concentration ranging, for example, from 0.05% to 3%, preferably from 0.1% to 1% and better still from 0.2% to 0.6% of the total weight of the emulsion.

Interestingly, the preparation of the simple inverse emulsion according to the invention has been found to require only a small amount of mechanical or thermal energy when compared with the preparations of other already known inverse emulsions.

The invention thus also features a method for preparing the compositions according to the invention. Specifically, the viscosity of the compositions according to the invention depends on the procedure. The procedure according to the invention includes two critical steps:
(i) pre-emulsification: the glycol must be incorporated slowly so as to ensure the formation of small globules, and the increase in stirring speed must be adapted;
(ii) the actual emulsification: the aqueous phase must be poured very slowly and with Rayneri stirring of 1,000 rpm.

Lastly, the final stirring must be very strict and of 30 minutes so as to be reproducible.

The process for preparing the compositions according to the invention comprises the following steps:
a) Preparation of the fatty phase A by:
weighing out the constituents of the fatty phase,
heating to 55° C.,
homogenization;
b) Preparation of the glycol or water-glycol phase B, with incorporation of the clobetasol 17-propionate, by:
weighing out the glycol(s) and the clobetasol 17-propionate,
magnetic stirring until the corticoid has dissolved, heating to 55° C.;
c) Pre-emulsification of the two phases prepared beforehand, by:
slow introduction of the glycol phase B into the fatty phase A,
increasing the stirring speed gradually as the propylene glycol is added, from an initial speed of 350 rpm to arrive at a speed of 1000 rpm, this final speed being kept constant for the rest of the formulation,
cooling the pre-emulsion obtained to room temperature;
d) Preparation of phase C, containing the viscosity-stabilizing compounds, by:
dissolving the surfactant electrolyte in water with stirring,
adding the viscosity stabilizer(s),
dispersing the viscosity stabilizer(s) in water with magnetic stirring;
e) Addition of phase D, containing the calcitriol, by:
preparing a stock solution of calcitriol in a suitable solvent,
adding an antioxidant,
stirring until the active agent has dissolved;
f) Mixing phase C and the required amount of phase D;

The term “required amount” means the amount of stock solution that makes it possible to obtain the desired amount of active agent in the final composition.
g) Emulsification of the mixture obtained in step f) with the pre-emulsion from step c) by very slowly introducing the aqueous phase C+D into the pre-emulsion obtained in step c), with Rayneri stirring at 1,000 rpm.

After the emulsification, stirring is continued for 30 minutes.

The present invention also features a regime or regimen for the administration or use of the subject novel inverse emulsions as described above in cosmetics and dermatology.

On account of the activity of the compounds used in the composition, the compositions according to the invention find application in the prevention and/or treatment of the following pathologies:
1) for treating dermatological complaints, conditions or afflictions associated with a differentiation or proliferation disorder of keratinocytes or sebocytes, especially for treating common acne, comedones, polymorphonuclear leukocytes, acne rosacea, nodulocystic acne, acne conglobata, senile acne and secondary acnes such as solar acne, acne medicamentosa or occupational acne;
2) for treating keratinization disorders, especially ichthyosis, ichthyosiform conditions, Darier's disease, palmoplantar keratoderma, leukoplakia and leukoplakiform conditions, and cutaneous or mucous (buccal) lichen;
3) for treating other dermatological complaints, conditions or afflictions associated with a keratinization disorder with an inflammatory and/or immunoallergic component, and especially all forms of psoriasis, whether cutaneous, mucous or ungual psoriasis, and even psoriatic rheumatism, or alternatively cutaneous atopy, such as eczema or respiratory atopy, or alternatively gingival hypertrophy;
4) for treating certain inflammatory skin afflictions not exhibiting a keratinization disorder, such as atopic eczema and contact allergies;
5) for treating all dermal or epidermal proliferations, whether benign or malignant, and whether of viral origin or not, such as common warts, flat warts and verruciform epidermodysplasia, oral or florid papillomatoses and proliferations that may be induced by ultraviolet radiation, especially in the case of basocellular and spinocellular epithelioma;
6) for treating other dermatological disorders such as bullous dermatosis and collagen diseases;
7) for preventing or treating the signs of aging of the skin, whether photo-induced or chronological aging, or for reducing actinic keratosis and pigmentations, or any cutaneous pathologies associated with chronological or actinic aging;
8) for preventing or treating cicatrization disorders or for preventing or repairing stretch marks;
9) for controlling sebaceous function disorders such as acneic hyperseborrhoea or simple seborrhoea or alternatively seborrhoeic eczema;
10) for treating certain ophthalmological disorders, especially corneopathies;
11) in the treatment or prevention of cancerous or precancerous skin conditions or other cancers presenting or being able to be induced to present vitamin D receptors, such as, but without limitation, breast cancer, leukemia, myelodysplasic syndromes and lymphomas, carcinomas of the cells of the malpighian epithelium and gastrointestinal cancers, melanomas and osteosarcoma;
12) in the treatment of inflammatory afflictions such as arthritis or rheumatoid arthritis;
13) in the treatment of any cutaneous or general disorder of viral origin;
14) in the prevention or treatment of alopecia of various origins, especially chemotherapy-induced or radiation-induced alopecia;
15) in the treatment of dermatological or general disorders having an immunological component;
16) in the treatment of immune disorders, such as autoimmune diseases (for instance, but without limitation, type 1 sugar diabetes, multiple sclerosis, lupus and lupus-type conditions, asthma, glomerulonephritis, etc.), selective dysfunctions of the immune system (for example AIDS) and the prevention of immune rejection [for instance graft rejections (for example kidney, heart, bone marrow, liver, pancreatic islets or the whole pancreas, the skin, etc.) or the prevention of graft-versus-host disease];
17) in the treatment of endocrine disorders, given that the vitamin D analogues can modulate hormonal secretion, such as increasing the secretion of insulin or selectively suppressing the secretion of parathyroid hormone (for example in chronic renal insufficiency and secondary hyperparathyroidism);
18) in the treatment of disorders characterized by abnormal management of intracellular calcium; and
19) in the treatment and/or prevention of vitamin D deficiencies and other disorders of homeostasis of minerals in the plasma and the bones, such as rickets, osteomalacia, osteoporosis, especially in the case of menopausal women, renal osteodystrophy and parathyroid function disorders.

This invention also features the pharmaceutical preparations and medicinal products formulated from the compositions according to the invention.

In particular, the invention thus relates to the use of a composition as defined above for the manufacture of a medicinal product for treating psoriasis.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Stabilities of Calcitriol in Various Excipients

Stability data for calcitriol in various excipients were generated.

a) Stability of Calcitriol in Ethanol:

30 ppm solution of calcitriol in qs 100% of absolute ethanol in the presence of 0.02% BHT.

HPLC assay technique against reference substance.

At the initial time (T0), it is considered that the composition comprises 100% calcitriol.

Calcitriol concentration measured as % relative to T0:

| Stability | | | | |
|---|---|---|---|---|
| conditions | T1 week | T2 weeks | T3 weeks | T4 weeks |
| −18° C. | 100.9% | 100.5% | 99.5% | 99.5% |
| +4° C. | 97.7% | 98.6% | 98.1% | 97.7% |
| +30° C. | / | 93.4% | / | 93.0% |

b) Stability of Calcitriol in Miglyol 812 (Caprylic/-15 Capric Triglycerides)

30 ppm solution of calcitriol in qs 100% Miglyol 812 in the presence of 0.4% BHT.

HPLC assay technique against reference substance.

At the initial time (T0), it is considered that the composition comprises 100% calcitriol.

Calcitriol concentration measured as % relative to T0:

| Stability conditions | T2 weeks | T4 weeks |
|---|---|---|
| +4° C. | 98.3% | 105.2% |
| RT | 95.1% | 98.0% |
| +40° C. | 91% | 93.8% |

These results show good stability of calcitriol in Miglyol 812.

c) Stability of Calcitriol in Cetiol SN (Cetearyl Isononanoate):

30 ppm solution of calcitriol in qs 100% Cetiol SN (cetearyl isononanoate) in the presence of 0.4% BHT.

HPLC assay technique against reference substance.

At the initial time (T0), it is considered that the composition comprises 100% calcitriol.

Calcitriol concentration measured as % relative to T0:

| Stability conditions | T2 weeks | T4 weeks |
|---|---|---|
| +4° C. | 98.6% | 98.1% |
| RT | 98.7% | 98.4% |
| +40° C. | 99.0% | 98.9% |

These results show good stability of calcitriol in Cetiol SN.

### EXAMPLE 2

### Viscosity Stabilization

The table below illustrates that the addition of the listed additives makes it possible to stabilize the viscosity over time. Three examples of formulations monitored over time are given:

| Chemical | Reference | Formulation | Formulation | Formulation |
|---|---|---|---|---|
| name | formulation | 1 | 2 | 3 |
| Oily phase | 16 | 16 | 16 | 16 |
| (mixture of | | | | |
| oils) | | | | |
| Propylene | qs 100% | qs 100% | qs 100% | qs 100% |
| glycol | | | | |
| Purified water | 14 | 14 | 14 | 14 |
| Magnesium | 1 | 1 | 1 | 1 |
| sulfate | | | | |
| heptahydrate | | | | |
| Butylhydroxy- | 0.1 | 0.1 | 0.1 | 0.1 |
| toluene | | | | |
| 95-96% | 5 | 5 | 5 | 5 |
| ethanol | | | | |
| Lauryl | 3 | | 3 | 3 |
| methicone | | | | |
| copolyol | | | | |
| Ceteareth-20 | 1 | | | |
| Cyclopenta- | | 4.5 | | |
| siloxane & | | | | |
| PEG/PPG- | | | | |
| 19/19 | | | | |
| dimethicone | | | | |
| Silicone | | 5 | | |
| elastomer and | | | | |
| decamethyl- | | | | |
| cyclopenta | | | | |
| siloxane | | | | |
| PEG-150 decyl | | | 1 | |
| alcohol/SMDI | | | | |
| copolymer | | | | |
| Glyceryl poly | | | | 5 |
| methacrylate | | | | |
| and propylene | | | | |
| glycol | | | | |

The flow threshold values are given in the following table:

| Temper- | Measuring | | Reference | Formulation | Formulation | Formulation |
|---|---|---|---|---|---|---|
| ature | time | | formulation | 1 | 2 | 3 |
| RT | 24 | h | 78 | 58 | 38 | 52 |
| | 1 | month | 81 | 47 | / | 49 |
| | 2 | months | 69 | / | 43 | 49 |
| | 3 | months | 50 | 60 | 39 | NC |

| | | | | |
|---|---|---|---|---|
| Conclusion | Drop in | Stable | Stable | Stable |
| | viscosity | | | |

The flow threshold of the reference formulation diminished over time, whereas formulations containing stabilizers are entirely stable over time.

The additives act by association by forming an emulsion-stabilizing network.

### EXAMPLE 3

### Protocol for Measuring the Stability of the Compositions According to the Invention

The physical stability of the formulations is measured by means of macroscopic and microscopic observation of the formulation at room temperature (RT), at 4° C. and at 40° C. after 15 days, 1 month, 2 months and 3 months.

At RT, the macroscopic observation makes it possible to ensure the physical integrity of the products and the microscopic observation makes it possible to check that there is no recrystallization of the dissolved active agent.

At 4° C., the microscopic observation checks that there is no recrystallization of the dissolved active agents.

At 40° C., the macroscopic observation checks the integrity of the finished product and the micro-scopic observation makes it possible to check the size stability of the globules.

The characterization of the finished product is completed by measuring the flow threshold. A Haake VT550 rheometer with an SVDIN measuring spindle is used. The rheograms are acquired at 25° C. and at a shear rate of 4 s⁻¹ (γ), and by measuring the shear stress. The term “flow threshold” (τ0) expressed in pascals) means the force (minimum shear stress) required to overcome the Van der Waals cohesion forces and to bring about flow. The flow threshold is likened to the value found at a shear rate of 4 s⁻¹.

These measurements are taken at T0 and after 15 days and 1, 2 and 3 months.

The examples that follow are examples of compositions according to the invention and the corresponding stability measurements thereof.

### EXAMPLE 4

### Composition

| | Amounts in |
|---|---|
| Starting materials | weight/weight % |
| Cyclopentasiloxane and | 4.5 |
| PEG/PPG-19/19 dimethicone | |
| Cyclomethicone 5 | 6 |
| Liquid paraffin | 3 |
| Cetostearyl isononanoate | 7 |
| Cyclomethicone 5/Dimethicone | 5 |
| Crosspolymer | |
| Clobetasol propionate | 0.001 |
| Propylene glycol | qs 100 |
| Purified water | 14 |
| Magnesium sulfate heptahydrate | 3 |
| Butylhydroxytoluene | 0.04 |
| Calcitriol | 0.0003 |
| 95% ethanol | 5 |

### EXAMPLE 5

### Composition

| | Amounts in |
|---|---|
| Starting materials | weight/weight % |
| Lauryl methicone copolyol | 3 |
| Cyclomethicone 5 | 6 |
| Liquid paraffin | 3 |
| Cetostearyl isononanoate | 7 |
| Clobetasol propionate | 0.05 |
| Propylene glycol | qs 100 |
| Ethylene/propylene/butylene/styrene | 5 |
| copolymer in isohexadecane | |
| Purified water | 14 |
| Magnesium sulfate heptahydrate | 3 |
| Butylhydroxytoluene | 0.04 |
| Calcitriol | 0.0009 |
| 95% ethanol | 5 |

### EXAMPLE 6

### Composition

| | Amounts in |
|---|---|
| Starting materials | weight/weight % |
| Lauryl methicone copolyol | 3 |
| Cyclomethicone 5 | 6 |
| Liquid paraffin | 3 |
| Cetostearyl isononanoate | 7 |
| Clobetasol propionate | 0.025 |
| Propylene glycol | qs 100 |
| Polyglyceryl methacrylate and | 5 |
| propylene glycol | |
| Purified water | 14 |
| Sorbitol | 8 |
| Magnesium sulfate heptahydrate | 3 |
| Butylhydroxytoluene | 0.04 |
| Calcitriol | 0.0003 |
| 95% ethanol\grave{ } | 5 |

Measurement of the Stabilities of the Composition According to the Protocol of Example 3:

SPECIFICATIONS AT T0

| τ0 | 152 | | Macroscopic | Translucent gel |
|---|---|---|---|---|
| | | | appearance | appearance, |
| | | | | firm and |
| | | | | shiny |
| Centrifugation | 3000 | no | Microscopic | Very fine |
| | rpm | comments | appearance | uniform |
| | 10,000 | no | | emulsion |
| | rpm | comments | | |

| RT | | | T 15 days |
|---|---|---|---|
| | Macroscopy | | In accordance |
| | Microscopy | | In accordance |
| | τ0 | | 176 |
| | Centrifugation | 3000 | No comments |
| | | 10,000 | No comments |

| | | |
|---|---|---|
| 4° C. | Macroscopy | In accordance |
| | Microscopy | In accordance |
| 40° C. | Macroscopy | In accordance |
| | Microscopy | |
| | τ0 | |

### EXAMPLE 7

### Composition

| | Amounts in |
|---|---|
| Starting materials | weight/weight % |
| Lauryl methicone copolyol | 3 |
| Cyclomethicone 5 | 6 |
| Liquid paraffin | 3 |
| Cetostearyl isononanoate | 7 |
| Clobetasol propionate | 0.025 |
| Propylene glycol | qs 100 |
| Polyglyceryl methacrylate | 5 |
| and propylene glycol | |
| Purified water | 14 |
| Glycerol | 8 |
| Magnesium sulfate heptahydrate | 3 |
| Butylhydroxytoluene | 0.04 |
| Calcitriol | 0.0003 |
| 95% ethanol | 5 |

### EXAMPLE 8

### Composition

| | Amounts in |
|---|---|
| Starting materials | weight/weight % |
| Lauryl methicone copolyol | 3 |
| Cyclomethicone 5 | 6 |
| Liquid paraffin | 3 |
| Cetostearyl isononanoate | 7 |
| Clobetasol propionate | 0.025 |
| Propylene glycol | qs 100 |
| Polyglyceryl methacrylate | 5 |
| and propylene glycol | |
| Purified water | 14 |
| PEG-150/decyl alcohol | 1 |
| Magnesium sulfate heptahydrate | 3 |
| Butylhydroxytoluene | 0.04 |
| Calcitriol | 0.0003 |
| 95% ethanol | 5 |

SPECIFICATIONS AT T0

| Tau 0 | | 85 | Macroscopic | Translucent, |
|---|---|---|---|---|
| | | | appearance | shiny gel |
| | | | | appearance |
| Centrifugation | 3000 | No | Very fine | Very fine |
| | rpm | comments | uniform | emulsion, no |
| | 10,000 | No | emulsion | crystals |
| | rpm | comments | | |

### EXAMPLE 9

### Composition

| | Amounts in |
|---|---|
| Starting materials | weight/weight % |
| Lauryl methicone copolyol | 3 |
| Cyclomethicone 5 | 5.5 |
| Liquid paraffin | 3 |
| Butylhydroxytoluene | 0.04 |
| Calcitriol | 0.0003 |
| Caprylic/capric triglyceride | 7.5 |
| Clobetasol propionate | 0.025 |
| Propylene glycol | qs 100 |
| Polyglyceryl methacrylate | 5 |
| and propylene glycol | |
| Purified water | 14 |
| PEG-150/decyl alcohol | 1 |
| Magnesium sulfate heptahydrate | 3 |
| 95% ethanol | 5 |

SPECIFICATIONS AT T0

| Tau 0 | | 82 | Macroscopic | Translucent, |
|---|---|---|---|---|
| | | | appearance | shiny gel |
| | | | | appearance |
| Centrifugation | 3000 | No | Very fine | Very fine |
| | rpm | comments | uniform | emulsion, no |
| | 10,000 | No | emulsion | crystals |
| | rpm | comments | | |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A topically applicable cosmetic/dermatological composition useful for the treatment of disorders of the skin, comprising an inverse emulsion which comprises a glycol or water-glycol dispersed hydrophilic phase, a lipophilic continuous phase, an emulsifier and having an HLB of between 2 and 7 and also having calcitriol and clobetasol 17-propionate dissolved therein.

2. The cosmetic/dermatological inverse emulsion as defined by claim 1, comprising at least one glycol and a minor amount of water.

3. The cosmetic/dermatological inverse emulsion as defined by claim 1, comprising from 0.00001% to 0.1% by weight of calcitriol.

4. The cosmetic/dermatological inverse emulsion as defined by claim 1, comprising from 0.0001% to 0.001% by weight of calcitriol.

5. The cosmetic/dermatological inverse emulsion as defined by claim 1, comprising from 0.0002% to 0.0005% by weight of calcitriol.

6. The cosmetic/dermatological inverse emulsion as defined by claim 1, comprising 0.0003% by weight of calcitriol.

7. The cosmetic/dermatological inverse emulsion as defined by claim 1, comprising from 0.0001% to 0.1% by weight of clobetasol 17-propionate.

8. The cosmetic/dermatological inverse emulsion as defined by claim 1, comprising from 0.001% to 0.05% by weight of clobetasol 17-propionate.

9. The cosmetic/dermatological inverse emulsion as defined by claim 1, comprising from 0.025% to 0.05% by weight of clobetasol 17-propionate.

10. The cosmetic/dermatological inverse emulsion as defined by claim 1, comprising calcitriol dissolved in an alcohol.

11. The cosmetic/dermatological inverse emulsion as defined by claim 10, comprising calcitriol dissolved in ethanol.

12. The cosmetic/dermatological inverse emulsion as defined by claim 1, comprising calcitriol dissolved in an oil.

13. The cosmetic/dermatological inverse emulsion as defined by claim 1, comprising calcitriol dissolved in caprylic/capric triglyceride.

14. The cosmetic/dermatological inverse emulsion as defined by claim 1, comprising clobetasol 17-propionate dissolved in the inner glycol or water-glycol phase.

15. The cosmetic/dermatological inverse emulsion as defined by claim 1, said glycol being selected from the group consisting of propylene glycol, hexylene glycol, dipropylene glycol and PEG-400.

16. The cosmetic/dermatological inverse emulsion as defined by claim 1, the weight proportion of glycol, relative to the total volume of the dispersed phase, ranging from 10% to 100%.

17. The cosmetic/dermatological inverse emulsion as defined by claim 1, the weight proportion of glycol, relative to the total weight of the composition, ranging from 20% to 90%.

18. The cosmetic/dermatological inverse emulsion as defined by claim 17, the weight proportion of glycol, relative to the total weight of the composition, ranging from 30% to 60%.

19. The cosmetic/dermatological inverse emulsion as defined by claim 1, said lipophilic phase comprising a mineral oil, a triglyceride, an ester, a silicone oil, or mixture thereof.

20. The cosmetic/dermatological inverse emulsion as defined by claim 19, comprising a liquid paraffin mineral oil.

21. The cosmetic/dermatological inverse emulsion as defined by claim 15, comprising caprylic/capric triglyceride.

22. The cosmetic/dermatological inverse emulsion as defined by claim 15, comprising cetearyl isononanoate.

23. The cosmetic/dermatological inverse emulsion as defined by claim 15, comprising a cyclomethicone.

24. The cosmetic/dermatological inverse emulsion as defined by claim 1, the weight proportion of the lipophilic phase, relative to the total weight of the composition, ranging from 5% to 60%.

25. The cosmetic/dermatological inverse emulsion as defined by claim 1, the weight proportion of the lipophilic phase, relative to the total weight of the composition, ranging from 10% to 25%.

26. The cosmetic/dermatological inverse emulsion as defined by claim 1, comprising a silicone emulsifier having an HLB of between 2 and 7.

27. The cosmetic/dermatological inverse emulsion as defined by claim 26, comprising a silicone emulsifier selected from the group consisting of lauryl methicone copolyol, cetyldimethicone copolyol, a mixture of dimethicone copolyol and cyclomethicone and a mixture of cetyldimethicone copolyol with polyglyceryl4 isostearate and hexyl laurate, or cyclopentasiloxane and PEG/PPG-19/19 dimethicone.

28. The cosmetic/dermatological inverse emulsion as defined by claim 26, comprising a cyclopentasiloxane and PEG/PPG-19/19 dimethicone or lauryl methicone copolyol silicone emulsifier.

29. The cosmetic/dermatological inverse emulsion as defined by claim 1, further comprising one or more viscosity stabilizers.

30. The cosmetic/dermatological inverse emulsion as defined by claim 29, further comprising at least one viscosity stabilizer selected from the group consisting of PEG-150 decyl alcohol/SMDI copolymer, polyglyceryl methacrylate and propylene glycol, the combination of ethylene/propylene/butylene/styrene copolymer in isododecane, the combination of ethylene/propylene/butylene/styrene copolymer in isohexadecane, dimethiconol behenate, silicone elastomer and decamethylcyclopentasiloxane.

31. The cosmetic/dermatological inverse emulsion as defined by claim 1, further comprising at least one electrolyte.

32. The cosmetic/dermatological inverse emulsion as defined by claim 1, further comprising at least one antioxidant compound.

33. The cosmetic/dermatological inverse emulsion as defined by claim 32, said at least one antioxidant comprising DL-α-tocopherol, butylhydroxyanisole or butylhydroxytoluene.

34. The cosmetic/dermatological inverse emulsion as defined by claim 1, the water activity of said dispersed hydrophilic phase being less than 0.85.

35. A cosmetic/dermatological composition suited for topical application onto the skin, integuments and/or mucous membranes of an individual afflicted with disorders thereof, comprising an inverse emulsion which comprises a glycol or water-glycol dispersed hydrophilic phase, from 55 to 60% of a lipophilic continuous phase, from 0.5% to 8% of an emulsifier having an HLB of between 2 and 7, and from 0.0001% to 1% by weight of calcitriol and 0.001% to 0.1% by weight of clobetasol 17-propionate dissolved therein, said inverse emulsion containing 30% to 100% of glycol and 1% to 30% of water.

36. A topically applicable cosmetic/dermatological composition useful for the treatment of disorders of the skin, integuments and/or mucous membranes, comprising a hydrophilic phase/lipophilic phase/hydrophilic phase triple emulsion and which further comprises an outer hydrophilic phase and a lipophilic phase constituting, with an inner hydrophilic phase, an inverse emulsion as defined by claim 1.

37. A process for the preparation of the cosmetic/dermatological inverse emulsion as defined by claim 1, comprising:
a) Preparation of a fatty phase A by:
weighing out the constituents of the fatty phase,
heating to 55° C.,
homogenization;
b) Preparation of a glycol or water-glycol phase B, with incorporation of the clobetasol 17-propionate, by:
weighing out the glycol(s) and the clobetasol 17-propionate,
magnetic stirring until the corticoid has dissolved,
heating to 55° C.;
c) Pre-emulsification of the two phases prepared above, by:
slow introduction of the glycol phase B into the fatty phase A,
increasing the stirring speed gradually as the glycol is added, from an initial speed of 350 rpm to a speed of 1000 rpm, this final speed being maintained constant for the remainder of the formulation,
cooling the pre-emulsion obtained to room temperature;
d) Preparation of a phase C, containing a viscosity-stabilizing compound, by:
dissolving a surfactant electrolyte in water with stirring,
adding the viscosity stabilizer(s),
dispersing the viscosity stabilizer(s) in water with magnetic stirring;
e) Addition of phase D, containing the calcitriol, by:
preparing a stock solution of calcitriol in a solvent,
adding an antioxidant,
stirring until the active agent has dissolved;
f) Mixing phase C and the required amount of phase D;
g) Emulsification of the mixture obtained in step f) with the pre-emulsion from step c) by very slowly introducing the aqueous phase C+D into the pre-emulsion obtained in step c), with Rayneri stirring at 1000 rpm.

38. A regime or regimen for preventing or treating dermatological conditions associated with a differentiation or proliferation disorder of keratinocytes or sebocytes; keratinization disorders; dermatological conditions associated with a keratinization disorder having an inflammatory and/or immunoallergic component; cutaneous inflammatory conditions not manifesting a keratinization disorder; dermal or epidermal proliferations; bullous dermatosis or collagen diseases; the signs of aging of the skin, or for reducing actinic keratoses and pigmentations, or any cutaneous pathologies associated with chronological or actinic aging; cicatrization disorders and stretch marks; acneic hyperseborrhoea or simple seborrhoea or seborrhoeic eczema; or dermatological disorders having an immunological component, comprising topically applying onto the skin, integuments and/or mucous membranes of an individual in need of such treatment, a thus effective amount of the cosmetic/dermatological inverse emulsion as defined by claim 1.

39. A regime or regimen for the treatment of psoriasis, comprising topically applying onto the affected area of the skin, integuments and/or mucous membranes of an individual afflicted with psoriasis, of a cosmetic/dermatological inverse emulsion as defined by claim 1.
